(19) United States
(12) Patent Application Publication
(10) Pub. No.: US 2012/0070457 A1
(43) Pub. Date: Mar. 22, 2012

(54) Polypeptides from neisseria meningitidis

(75) Inventors: Sean C. Daugherty, Rosedale, MD (US); Julie C. Dunning Hotopp, Columbia, MD (US); David R. Riley, Rosedale, MD (US); Samuel V. Angiuoli, Arlington, VA (US); Heather Huot Creasy, Gaithersburg, MD (US); Todd H. Creasy, Gaithersburg, MD (US); Herve Tettelin, Ellicott City, MD (US); Duccio Medini, Perugia (IT); Emilio Siena, Colle Val D'Elsa (IT); Sonia Budroni, Martis (IT); Maria Scarselli, Siena (IT)

(73) Assignees: J. CRAIG VENTER INSTITUTE, INC., Rockville, MD (US); NOVARTIS AG, Basel (CH)

(21) Appl. No.: 13/199,782

(22) Filed: Sep. 8, 2011

Related U.S. Application Data
(60) Provisional application No. 61/403,103, filed on Sep. 10, 2010.

(51) Int. Cl. A61K 39/095; A61P 31/04; C07K 16/12; C07H 21/04; C07K 14/22; C07K 7/06

(52) U.S. Cl. 424/190.1; 530/350; 530/329; 530/387.9; 536/23.7; 536/23.53

(57) ABSTRACT

Polypeptides comprising Neisseria meningitidis amino acid sequences. Over 36000 meningococcal polypeptide sequences are disclosed. The invention also provides related polypeptides, nucleic acids, antibodies and methods. These can all be usedin medicine for treating or preventing disease and/or infection caused by N. meningitidis, such as meningococcal meningitis.

### CROSS REFERENCE TO RELATED APPLICATIONS

This application claims the benefit under 35 USC 119(e) of prior copending U.S. Provisional Patent Application No. 61/403,103, filed Sep. 10, 2010, the disclosure of which is hereby incorporated by reference in its entirety.

### TECHNICAL FIELD

This invention is in the field of Neisseria meningitidis immunology and vaccinology.

### INCORPORATION BY REFERENCE OF SEQUENCE LISTING ON ASCII TEXT FILE

The content of the following submission on ASCII text file is incorporated herein by reference in its entirety: a computer readable form (CRF) of the Sequence Listing (file name: 223002118400Sequencelisting.txt, date recorded: Sep. 2, 2011, size: 136,436 KB).

### INCORPORATION BY REFERENCE OF TABLE PROVIDED ON COMPACT DISCS

The content of the following submission on ASCII text file is incorporated herein by reference in its entirety: a computer readable form (CRF) of Table 1 (file name: 223002118400Table1.txt, date recorded: Sep. 2, 2011, size: 1,297 KB).

LENGTHY TABLES
The patent application contains a lengthy table section. A copy of the table is available in electronic form from the USPTO web site (http://seqdata.uspto.gov/?pageRequest=docDetail&DocID=US20120070457A1). An electronic copy of the table will also be available from the USPTO upon request and payment of the fee set forth in 37 CFR 1.19(b)(3).

### BACKGROUND ART

Neisseria meningitidis is a non-motile Gram-negative diplococcus that is pathogenic in humans. It colonises the pharynx and causes meningitis (and, occasionally, septicaemia in the absence of meningitis). Several genome sequences for meningococcus have been published (e.g. refs 1 & 2) and these have been mined to identify vaccine candidates. This approach has successfully led to the “universal vaccine” disclosed in reference 3.

It is an object of the invention to provide further and better polypeptides for use in the development of meningococcal vaccines. The polypeptides may also be useful for diagnostic purposes, and as targets for antibiotics.

### DISCLOSURE OF THE INVENTION

### Polypeptides

The invention provides polypeptides comprising the N. meningitidis amino acid sequences disclosed in the sequence listing as SEQ ID NOs: 1 to 36178 and 72357 to 72990.

The invention also provides polypeptides comprising amino acid sequences that have sequence identity to the N. meningitidis amino acid sequences disclosed in the sequence listing. The degree of sequence identity should be greater than 50% (e.g. 60%, 70%, 75%, 80%, 85%, 90%, 91%, 92%, 93%, 94%, 95%, 96%, 97%, 98%, 99% or more). These polypeptides include homologs, orthologs, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty=12 and gap extension penalty=1.

These polypeptides may, compared to the meningococcal sequences of the sequence listing, include one or more (e.g. 1, 2, 3, 4, 5, 6, 7, 8, 9, 10, etc.; ideally fewer than 20) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cystine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, 10, etc.) single amino acid deletions relative to the meningococcal sequences of the sequence listing. The polypeptides may also include one or more (e.g. 1, 2, 3, 4, 5, 6, 7, 8, 9, 10, etc.) insertions (e.g. each of 1, 2, 3, 4 or 5 amino acids) relative to the meningococcal sequences of the sequence listing.

The invention further provides polypeptides comprising fragments of the N. meningitidis amino acid sequences of the sequence listing. The fragments should comprise at least n consecutive amino acids from the sequences, where n is 6 or more (e.g. 7, 8, 9, 10, 11, 12, 13, 14, 15, 16, 17, 18, 19, 20, 22, 24, 26, 28, 30, 35, 40, 45, 50, 60, 70, 80, 90, 100 or more). Preferred fragments (particularly those which are at least 10 amino acids long) are those which are present in more than one sequence within SEQ ID NOs: 1 to 36178 or 72357 to 72990. For instance, amino acids 217-240 of SEQ ID NO: 15 are also present in SEQ ID NOs: 2253, 4529, 7532, 8506, 11119, 13289, 15743, 17631, 18199, 20589, 23114, 24374, 26153, 29120, 31620, 34033, and 34531, so this is a preferred fragment of SEQ ID NO: 15 (and, of course, also of each of SEQ ID NOs: 2253, 4529, 7532, 8506, 11119, 13289, 15743, 17631, 18199, 20589, 23114, 24374, 26153, 29120, 31620, 34033, and 34531).

The fragment may comprise at least one T-cell or, preferably, a B-cell epitope of the sequence. T- and B-cell epitopes can be identified empirically (e.g. using PEPSCAN [4,5] or similar methods), or they can be predicted (e.g. using the Jameson-Wolf antigenic index [6], matrix-based approaches [7], TEPITOPE [8], neural networks [9], OptiMer & EpiMer [10, 11], ADEPT [12], Tsites [13], hydrophilicity [14], antigenic index [15] or the methods disclosed in reference 16 etc.). Other preferred fragments are (a) the N-terminal leader peptides of the meiningococcal polypeptides of the invention, (b) the meningococcal polypeptides, but without their N-terminal leader peptides, (c) the meningococcal polypeptides, but without their N-terminal amino acid residue.

Where the invention uses an amino acid sequence which has less than 100% identity to an amino acid sequence in the sequence listing, or where it includes a fragment of an amino acid sequence in the sequence listing, it is preferred that the polypeptide of the invention should be able to bind to an antibody which binds specifically to a protein that consists of the relevant amino acid sequence from the sequence listing. For example, a variant of SEQ ID NO: 1 would still be able to bind to an antibody which was raised against SEQ ID NO: 1. As used herein, an antibody “binds specifically” to a protein when its binding affinity for that protein is at least 100-fold tighter than for human serum albumin i.e. the binding is not non-specific.

Preferred amino acid SEQ ID NOs of the invention are listed in Table 2, including polypeptides that are lipidated, that are located in the outer membrane, that are adhesins, that are located in the inner membrane, or that are located in the periplasm.

Particualrly preferred amino acid SEQ ID NOs of the invention are listed in Table 3.

Polypeptides of the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [17,18]. Solid-phase peptide synthesis is particularly preferred, such as methods based on tBoc or Fmoc [19] chemistry. Enzymatic synthesis [20] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [21]. Where D-amino acids are included, however, it is preferred to use chemical synthesis. Polypeptides of the invention may have covalent modifications at the C-terminus and/or N-terminus.

Polypeptides of the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides of the invention are preferably provided in purified or substantially purified form i.e. substantially free from other polypeptides (e.g. free from naturally-occurring polypeptides), particularly from other meningococcal or host cell polypeptides, and are generally at least about 50% pure (by weight), and usually at least about 90% pure i.e. less than about 50%, and more preferably less than about 10% (e.g. 5%) of a composition is made up of other expressed polypeptides. Polypeptides of the invention are preferably meningococcal polypeptides. Polypeptides of the invention preferably have the property indicated in Table 1 for the relevant sequence.

Polypeptides of the invention may be attached to a solid support. Polypeptides of the invention may comprise a detectable label (e.g. a radioactive or fluorescent label, or a biotin label).

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

The invention provides polypeptides comprising sequence -X-Y- or -Y-X-, wherein: -X- is the amino acid sequence of an polypeptide as defined above and -Y- is not an amino acid sequence of a polypeptide as defined above i.e. the invention provides fusion proteins. Where the N-terminus codon of the downstream polypeptide-coding sequence is not ATG then that codon will be translated as the standard amino acid for that codon rather than as a Met, which occurs when the codon is a start codon. The -Y- sequence will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct polypeptide trafficking, or short peptide sequences which facilitate cloning or purification (e.g. histidine tags i.e. Hisₙ where n=3, 4, 5, 6, 7, 8, 9, 10 or more).

The invention provides a process for producing polypeptides of the invention, comprising the step of culturing a host cell of to the invention under conditions which induce polypeptide expression.

The invention provides a process for producing a polypeptide of the invention, wherein the polypeptide is synthesised in part or in whole using chemical means.

The invention provides a composition comprising two or more polypeptides of the invention.

The invention also provides a hybrid polypeptide represented by the formula NH₂-A-[—X-L-]ₙ-B-COOH, wherein X is a polypeptide of the invention as defined above, L is an optional linker amino acid sequence, A is an optional N-terminal amino acid sequence, B is an optional C-terminal amino acid sequence, and n is an integer greater than 1. The value of n is between 2 and x, and the value of x is typically 3, 4, 5, 6, 7, 8, 9 or 10. Preferably n is 2, 3 or 4; it is more preferably 2 or 3; most preferably, n=2. For each n instances, -X- may be the same or different. For each n instances of [—X-L-], linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 19, 18, 17, 16, 15, 14, 13, 12, 11, 10, 9, 8, 7, 6, 5, 4, 3, 2, 1). Examples include short peptide sequences which facilitate cloning, poly-glycine linkers (i.e. Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. -A- and -B- are optional sequences which will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct polypeptide trafficking, or short peptide sequences which facilitate cloning or purification (e.g. histidine tags i.e. Hisₙ where n=3, 4, 5, 6, 7, 8, 9, 10 or more). Other suitable N-terminal and C-terminal amino acid sequences will be apparent to those skilled in the art.

Various tests can be used to assess the in vivo immunogenicity of polypeptides of the invention. For example, polypeptides can be expressed recombinantly and used to screen patient sera by immunoblot. A positive reaction between the polypeptide and patient serum indicates that the patient has previously mounted an immune response to the protein in question i.e. the protein is an immunogen. This method can also be used to identify immunodominant proteins.

### Antibodies

The invention provides antibodies that bind to polypeptides of the invention. These may be polyclonal or monoclonal and may be produced by any suitable means (e.g. by recombinant expression). To increase compatibility with the human immune system, the antibodies may be chimeric or humanised [e.g. refs. 22 & 23], or fully human antibodies may be used. The antibodies may include a detectable label (e.g. for diagnostic assays). Antibodies of the invention may be attached to a solid support. Antibodies of the invention are preferably neutralising antibodies.

Monoclonal antibodies are particularly useful in identification and purification of the individual polypeptides against which they are directed. Monoclonal antibodies of the invention may also be employed as reagents in immunoassays, radioimmunoassays (RIA) or enzyme-linked immunosorbent assays (ELISA), etc. In these applications, the antibodies can be labelled with an analytically-detectable reagent such as a radioisotope, a fluorescent molecule or an enzyme. The monoclonal antibodies produced by the above method may also be used for the molecular identification and characterization (epitope mapping) of polypeptides of the invention.

Antibodies of the invention are preferably provided in purified or substantially purified form. Typically, the antibody will be present in a composition that is substantially free of other polypeptides e.g. where less than 90% (by weight), usually less than 60% and more usually less than 50% of the composition is made up of other polypeptides.

Antibodies of the invention can be of any isotype (e.g. IgA, IgG, IgM i.e. an α, γ or μ heavy chain), but will generally be IgG. Within the IgG isotype, antibodies may be IgG1, IgG2, IgG3 or IgG4 subclass. Antibodies of the invention may have a κ or a λ light chain.

Antibodies of the invention can take various forms, including whole antibodies, antibody fragments such as F(ab′)2 and F(ab) fragments, Fv fragments (non-covalent heterodimers), single-chain antibodies such as single chain Fv molecules (scFv), minibodies, oligobodies, etc. The term “antibody” does not imply any particular origin, and includes antibodies obtained through non-conventional processes, such as phage display.

The invention provides a process for detecting polypeptides of the invention, comprising the steps of: (a) contacting an antibody of the invention with a biological sample under conditions suitable for the formation of an antibody-antigen complexes; and (b) detecting said complexes.

The invention provides a process for detecting antibodies of the invention, comprising the steps of: (a) contacting a polypeptide of the invention with a biological sample (e.g. a blood or serum sample) under conditions suitable for the formation of an antibody-antigen complexes; and (b) detecting said complexes.

### Nucleic Acids

The invention provides nucleic acid comprising the N. meningitidis nucleotide sequences of the sequence listing. These nucleic acid sequences are SEQ ID NOs: 36179-72356.

The invention also provides nucleic acid comprising nucleotide sequences having sequence identity to the N. meningitidis nucleotide sequences of the sequence listing. Identity between sequences is preferably determined by the Smith-Waterman homology search algorithm as described above.

The invention also provides nucleic acid which can hybridize to the N. meningitidis nucleic acid of the sequence listing. Hybridization reactions can be performed under conditions of different “stringency”. Conditions that increase stringency of a hybridization reaction of widely known and published in the art (e.g. page 7.52 of reference 24). Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [e.g. see references 24-27, etc.].

In some embodiments, nucleic acid of the invention hybridizes to a target of the invention under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

Nucleic acid comprising fragments of these sequences are also provided. These should comprise at least n consecutive nucleotides from the N. meningitidis sequences, wherein n is 10 or more (e.g. 12, 14, 15, 18, 20, 25, 30, 35, 40, 50, 60, 70, 80, 90, 100, 150, 200 or more).

The invention provides nucleic acid of formula 5′-X-Y-Z-3′, wherein: -X- is a nucleotide sequence consisting of x nucleotides; -Z- is a nucleotide sequence consisting of z nucleotides; -Y- is a nucleotide sequence consisting of either (a) a fragment of one of SEQ ID NOs: 36179-72356, or (b) the complement of (a); and said nucleic acid 5′-X-Y-Z-3′ is neither (i) a fragment of one of SEQ ID NOs: 36179-72356 nor (ii) the complement of (i). The -X- and/or -Z- moieties may comprise a promoter sequence (or its complement).

The invention also provides nucleic acid encoding the polypeptides and polypeptide fragments of the invention.

The invention includes nucleic acid comprising sequences complementary to the sequences disclosed in the sequence listing (e.g. for antisense or probing, or for use as primers), as well as the sequences in the orientation actually shown.

Nucleic acids of the invention can be used in hybridisation reactions (e.g. Northern or Southern blots, or in nucleic acid microarrays or ‘gene chips’) and amplification reactions (e.g. PCR, SDA, SSSR, LCR, TMA, NASBA, etc.) and other nucleic acid techniques.

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other N. meningitidis or host cell nucleic acids, generally being at least about 50% pure (by weight), and usually at least about 90% pure. Nucleic acids of the invention are preferably meningococcal nucleic acids.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention which comprise meningococcal sequences may also comprise non-meningococcal sequences (e.g. in nucleic acids of formula 5′-X-Y-Z-3′, as defined above). This is particularly useful for primers, which may thus comprise a first sequence complementary to a PCAV nucleic acid target and a second sequence which is not complementary to the nucleic acid target. Any such non-complementary sequences in the primer are preferably 5′ to the complementary sequences. Typical non-complementary sequences comprise restriction sites or promoter sequences.

Nucleic acids of the invention can be prepared in many ways e.g. by chemical synthesis (at least in part), by digesting longer nucleic acids using nucleases (e.g. restriction enzymes), by joining shorter nucleic acids (e.g. using ligases or polymerases), from genomic or cDNA libraries, etc.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T). The terms also imply a direction—the complement of 5′-ACAGT-3′ is 5′-ACTGT-3′ rather than 5′-TGTCA-3′.

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

The invention also provides a kit comprising primers (e.g. PCR primers) for amplifying a template sequence contained within a Neisseria bacterium (e.g. N. meningitidis) nucleic acid sequence, the kit comprising a first primer and a second primer, wherein the first primer is substantially complementary to said template sequence and the second primer is substantially complementary to a complement of said template sequence, wherein the parts of said primers which have substantial complementarity define the termini of the template sequence to be amplified. The first primer and/or the second primer may include a detectable label (e.g. a fluorescent label).

The invention also provides a kit comprising first and second single-stranded oligonucleotides which allow amplification of a Neisseria template nucleic acid sequence contained in a single- or double-stranded nucleic acid (or mixture thereof), wherein: (a) the first oligonucleotide comprises a primer sequence which is substantially complementary to said template nucleic acid sequence; (b) the second oligonucleotide comprises a primer sequence which is substantially complementary to the complement of said template nucleic acid sequence; (c) the first oligonucleotide and/or the second oligonucleotide comprise(s) sequence which is not compementary to said template nucleic acid; and (d) said primer sequences define the termini of the template sequence to be amplified. The non-complementary sequence(s) of feature (c) are preferably upstream of (i.e. 5′ to) the primer sequences. One or both of these (c) sequences may comprise a restriction site [e.g. ref. 28] or a promoter sequence [e.g. 29]. The first oligonucleotide and/or the second oligonucleotide may include a detectable label (e.g. a fluorescent label).

The template sequence may be any part of a genome sequence.

The invention provides a process for detecting nucleic acid of the invention, comprising the steps of: (a) contacting a nucleic probe according to the invention with a biological sample under hybridising conditions to form duplexes; and (b) detecting said duplexes.

The invention provides a process for detecting N. meningitidis in a biological sample (e.g. blood), comprising the step of contacting nucleic acid according to the invention with the biological sample under hybridising conditions. The process may involve nucleic acid amplification (e.g. PCR, SDA, SSSR, LCR, TMA, NASBA, etc.) or hybridisation (e.g. microarrays, blots, hybridisation with a probe in solution etc.). PCR detection of N. meningitidis in clinical samples has been reported (e.g. see refs. 30 & 31). Clinical assays based on nucleic acid are described in general in ref. 32.

The invention provides a process for preparing a fragment of a target sequence, wherein the fragment is prepared by extension of a nucleic acid primer. The target sequence and/or the primer are nucleic acids of the invention. The primer extension reaction may involve nucleic acid amplification (e.g. PCR, SDA, SSSR, LCR, TMA, NASBA, etc.).

Nucleic acid amplification according to the invention may be quantitative and/or real-time.

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

### Pharmaceutical Compositions

The invention provides compositions comprising: (a) polypeptide, antibody, and/or nucleic acid of the invention; and (b) a pharmaceutically acceptable carrier. These compositions may be suitable as immunogenic compositions, for instance, or as diagnostic reagents, or as vaccines. Vaccines according to the invention may either be prophylactic (i.e. to prevent infection) or therapeutic (i.e. to treat infection), but will typically be prophylactic.

A ‘pharmaceutically acceptable carriers’ includes any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in ref. 161.

Compositions of the invention may include an antimicrobial, particularly if packaged in a multiple dose format.

Compositions of the invention may comprise detergent e.g. a Tween (polysorbate), such as Tween 80. Detergents are generally present at low levels e.g. <0.01%.

Compositions of the invention may include sodium salts (e.g. sodium chloride) to give tonicity. A concentration of 10±2 mg/ml NaCl is typical.

Compositions of the invention will generally include a buffer. A phosphate buffer is typical.

Compositions of the invention may comprise a sugar alcohol (e.g. mannitol) or a disaccharide (e.g. sucrose or trehalose) e.g. at around 15-30 mg/ml (e.g. 25 mg/ml), particularly if they are to be lyophilised or if they include material which has been reconstituted from lyophilised material. The pH of a composition for lyophilisation may be adjusted to around 6.1 prior to lyophilisation.

Polypeptides of the invention may be administered in conjunction with other immunoregulatory agents. In particular, compositions will usually include a vaccine adjuvant. Adjuvants which may be used in compositions of the invention include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. (e.g. see chapters 8 & 9 of ref. 33), or mixtures of different mineral compounds, with the compounds taking any suitable form (e.g. gel, crystalline, amorphous, etc.), and with adsorption being preferred. The mineral containing compositions may also be formulated as a particle of metal salt [34].

A typical adjuvant is amorphous aluminium hydroxyphosphate with PO₄/Al molar ratio between 0.84 and 0.92, included at 0.6 mg A1³⁺/ml. Adsorption with a low dose of aluminium phosphate may be used e.g. between 50 and 100 μg Al³⁺ per conjugate per dose. Where there is more than one conjugate in a composition, not all conjugates need to be adsorbed.

### B. Oil Emulsions

Oil emulsion compositions suitable for use as adjuvants in the invention include squalene-in-water emulsions, such as MF59 (Chapter 10 of ref. 33; see also ref. 35; 5% Squalene, 0.5% Tween 80, and 0.5% Span 85, formulated into submicron particles using a microfluidizer) or AS03. Complete Freund's adjuvant (CFA) and incomplete Freund's adjuvant (IFA) may also be used.

### C. Saponin Formulations [Chapter 22 of Ref. 33]

Saponin formulations may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 36. Saponin formulations may also comprise a sterol, such as cholesterol [37].

Combinations of saponins and cholesterols can be used to form unique particles called immunostimulating complexs (ISCOMs) [chapter 23 of ref. 33]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 37-39. Optionally, ISCOMS may be devoid of additional detergent [40].

A review of the development of saponin based adjuvants can be found in refs. 41 & 42.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 43-48. Virosomes are discussed further in, for example, ref. 49

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 50. Such “small particles” of 3dMPL are small enough to be sterile filtered through a 0.22 μm membrane [50]. Other non-toxic LPS derivatives include monophosphoryl lipid A mimics, such as aminoalkyl glucdsaminide phosphate derivatives e.g. RC-529 [51,52].

Lipid A derivatives include derivatives of lipid A from Escherichia coli such as OM-174. OM-174 is described for example in refs. 53 & 54.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 55, 56 and 57 disclose possible analog substitutions e.g. replacement of guanosine with 2′-deoxy-7-deazaguanosine. The adjuvant effect of CpG oligonucleotides is further discussed in refs. 58-63.

The CpG sequence may be directed to TLR9, such as the motif GTCGTT or TTCGTT [64]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 65-67. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 64 & 68-70.

Bacterial ADP-ribosylating toxins and detoxified derivatives thereof may be used as adjuvants in the invention. Preferably, the protein is derived from E.coli (E.coli heat labile enterotoxin “LT”), cholera (“CT”), or pertussis (“PT”). The use of detoxified ADP-ribosylating toxins as mucosal adjuvants is described in ref. 71 and as parenteral adjuvants in ref. 72. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivaties thereof, particularly LT-K63 and LT-R72, as adjuvants can be found in refs. 73-80. Numerical reference for amino acid substitutions is preferably based on the alignments of the A and B subunits of ADP-ribosylating toxins set forth in ref. 81, specifically incorporated herein by reference in its entirety.

F. Human Immunomodulators Human immunomodulators suitable for use as adjuvants in the invention include cytokines, such as interleukins (e.g. IL-1, IL-2, IL-4, IL-5, IL-6, IL-7, IL-12 [82], etc.) [83], interferons (e.g. interferon-γ), macrophage colony stimulating factor, and tumor necrosis factor.

### G. Bioadhesives and Mucoadhesives

Bioadhesives and mucoadhesives may also be used as adjuvants in the invention. Suitable bioadhesives include esterified hyaluronic acid microspheres [84] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [85].

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(a-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of Ref 33)

Examples of liposome formulations suitable for use as adjuvants are described in refs. 86-88.

### J. Polyoxyethylene Ether and Polyoxyethylene Ester Formulations

Adjuvants suitable for use in the invention include polyoxyethylene ethers and polyoxyethylene esters [89]. Such formulations further include polyoxyethylene sorbitan ester surfactants in combination with an octoxynol [90] as well as polyoxyethylene alkyl ethers or ester surfactants in combination with at least one additional non-ionic surfactant such as an octoxynol [91]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Polyphosphazene (PCPP)

PCPP formulations are described, for example, in refs. 92 and 93.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e,g. “Resiquimod 3M”), described further in refs. 94 and 95.

The invention may also comprise combinations of aspects of one or more of the adjuvants identified above. For example, the following adjuvant compositions may be used in the invention: (1) a saponin and an oil-in-water emulsion [96]; (2) a saponin (e.g. QS21) +a non-toxic LPS derivative (e.g. 3dMPL) [97]; (3) a saponin (e.g. QS21) +a non-toxic LPS derivative (e.g. 3dMPL) +a cholesterol; (4) a saponin (e.g. QS21) +3dMPL +IL-12 (optionally +a sterol) [98]; (5) combinations of 3dMPL with, for example, QS21 and/or oil-in-water emulsions [99]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL +CWS (Detox™); and (8) one or more mineral salts (such as an aluminum salt) +a non-toxic derivative of LPS (such as 3dMPL).

Other substances that act as immunostimulating agents are disclosed in chapter 7 of ref. 33.

The use of an aluminium hydroxide or aluminium phosphate adjuvant is particularly preferred, and antigens are generally adsorbed to these salts. Calcium phosphate is another preferred adjuvant.

The pH of compositions of the invention is preferably between 6 and 8, preferably about 7. Stable pH may be maintained by the use of a buffer. Where a composition comprises an aluminium hydroxide salt, it is preferred to use a histidine buffer [100]. The composition may be sterile and/or pyrogen-free. Compositions of the invention may be isotonic with respect to humans.

Compositions may be presented in vials, or they may be presented in ready-filled syringes. The syringes may be supplied with or without needles. A syringe will include a single dose of the composition, whereas a vial may include a single dose or multiple doses. Injectable compositions will usually be liquid solutions or suspensions. Alternatively, they may be presented in solid form (e.g. freeze-dried) for solution or suspension in liquid vehicles prior to injection.

Compositions of the invention may be packaged in unit dose form or in multiple dose form. For multiple dose forms, vials are preferred to pre-filled syringes. Effective dosage volumes can be routinely established, but a typical human dose of the composition for injection has a volume of 0.5 ml.

Where a composition of the invention is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials, and a typical quantity of each antigen per dose is between 1 μg and 10 mg.

### Pharmaceutical Uses

The invention also provides a method of treating a patient, comprising administering to the patient a therapeutically effective amount of a composition of the invention. The patient may either be at risk from the disease themselves or may be a pregnant woman (‘maternal immunisation’).

The invention provides nucleic acid, polypeptide, or antibody of the invention for use as medicaments (e.g. as immunogenic compositions or as vaccines) or as diagnostic reagents. It also provides the use of nucleic acid, polypeptide, or antibody of the invention in the manufacture of: (i) a medicament for treating or preventing disease and/or infection caused by N. meningitidis; (ii) a diagnostic reagent for detecting the presence of N. meningitidis or of antibodies raised against N. meningitidis; and/or (iii) a reagent which can raise antibodies against N. meningitidis. Said N. meningitidis serotype or strain, but is preferably a non-typeable N. meningitidis. Said disease may be, for instance, bacterial meningitis, meningococcal meningitis, septicaemia, meningococcemia, Waterhouse-Friderichsen syndrome, adrenal insufficiency, or disseminated intravascular coagulation.

The patient is preferably a human. Where the vaccine is for prophylactic use, the human is preferably a child (e.g. a toddler or infant); where the vaccine is for therapeutic use, the human is preferably an adult. A vaccine intended for children may also be administered to adults e.g. to assess safety, dosage, immunogenicity, etc.

One way of checking efficacy of therapeutic treatment involves monitoring meningococcal infection after administration of the composition of the invention. One way of checking efficacy of prophylactic treatment involves monitoring immune responses against an administered polypeptide after administration. Immunogenicity of compositions of the invention can be determined by administering them to test subjects (e.g. children 12-16 months age, or animal models e.g. mice, as used in a typical serum bactericidal assay) and then determining standard parameters including ELISA titres (GMT) of IgG. These immune responses will generally be determined around 4 weeks after administration of the composition, and compared to values determined before administration of the composition. Where more than one dose of the composition is administered, more than one post-administration determination may be made.

Administration of polypeptide antigens is a preferred method of treatment for inducing immunity. Administration of antibodies of the invention is another preferred method of treatment. This method of passive immunisation is particularly useful for newborn children or for pregnant women. This method will typically use monoclonal antibodies, which will be humanised or fully human.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml.

The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

Bacterial infections affect various areas of the body and so compositions may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition). The composition may be prepared for topical administration e.g. as an ointment, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder [e.g. refs 101 & 102].

### Vesicle-Containing Compositions

An immunogenic composition of the invention may include a meningococcal outer membrane vesicle e.g. in combination with a polypeptide of the invention. In some embodiments, a vesicle and a polypeptide of the invention can be prepared separately, and then mixed to give the immunogenic composition [118]. In other embodiments, outer membrane vesicles can be prepared from a recombinant meningococcus that hyper-expresses the polypeptide of the invention. The invention also provides such hyper-expressing strains. The polypeptide expressed by these strains should be in an immunoaccessible form in the vesicles.

The outer membrane vesicles include any proteoliposomic vesicle obtained by disruption of or blebbling from a meningococcal outer membrane to form vesicles therefrom that include protein components of the outer membrane. Thus the term includes OMVs (sometimes referred to as ‘blebs’), microvesicles (MVs [103]) and ‘native OMVs’ (‘NOMVs’ [104]).

MVs and NOMVs are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 105 & 106 describe Neisseria with high MV production.

OMVs are prepared artificially from bacteria, and may be prepared using detergent treatment (e.g. with deoxycholate), or by non-detergent means (e.g. see reference 107). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [108 & 109] being preferred for treating Neisseria) at a pH sufficiently high not to precipitate the detergent [110]. Other techniques may be performed substantially in the absence of detergent [107] using techniques such as sonication, homogenisation, microfluidisation, cavitation, osmotic shock, grinding, French press, blending, etc. Methods using no or low detergent can retain useful antigens such as NspA [107]. Thus a method may use an OMV extraction buffer with about 0.5% deoxycholate or lower e.g. about 0.2%, about 0.1%, <0.05% or zero.

A useful process for OMV preparation is described in reference 111 and involves ultrafiltration on crude OMVs, rather than instead of high speed centrifugation. The process may involve a step of ultracentrifugation after the ultrafiltration takes place.

Vesicles for use with the invention can be prepared from any meningococcal strain. The vesicles will usually be from a serogroup B strain, but it is possible to prepare them from serogroups other than B (e.g. reference 110 discloses a process for serogroup A), such as A, C, W135 or Y. The strain may be of any serotype (e.g. 1, 2a, 2b, 4, 14, 15, 16, etc.), any serosubtype, and any immunotype (e.g. L1; L2; L3; L3,3,7; L10; etc.). The meningococci may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3. These lineages have been defined by multilocus enzyme electrophoresis (MLEE), but multilocus sequence typing (MLST) has also been used to classify meningococci [ref. 112] e.g. the ET-37 complex is the ST-11 complex by MLST, the ET-5 complex is ST-32 (ET-5), lineage 3 is ST-41/44, etc. Vesicles can be prepared from strains having one of the following subtypes: P1.2; P1.2,5; P1.4; P1.5; P1.5,2; P1.5,c; P1.5c,10; P1.7,16; P1.7,16b; P1.7h,4; P1.9; P1.15; P1.9,15; P1.12,13; P1.13; P1.14; P1.21,16; P1.22,14.

Vesicles used with the invention may be prepared from wild-type meningococcal strains or from mutant meningococcal strains (including strains engineered to hyper-express a polypeptide of the invention). For instance, reference 113 discloses preparations of vesicles obtained from N. meningitidis with a modified fur gene. Reference 121 teaches that nspA expression should be up-regulated with concomitant porA and cps knockout. Further knockout mutants of N. meningitidis for OMV production are disclosed in references 121 to 123. Reference 114 discloses vesicles in which fHBP is upregulated. Reference 115 discloses the construction of vesicles from strains modified to express six different PorA subtypes. Mutant Neisseria with low endotoxin levels, achieved by knockout of enzymes involved in LPS biosynthesis, may also be used [116,117]. These or others mutants can all be used with the invention.

Thus a strain used with the invention may in some embodiments express more than one PorA subtype. 6-valent and 9-valent PorA strains have previously been constructed. The strain may express 2, 3, 4, 5, 6, 7, 8 or 9 of PorA subtypes: P1.7,16; P1.5-1,2-2; P1.19,15-1; P1.5-2,10; P1.12-1,13; P1.7-2,4; P1.22,14; P1.7-1,1 and/or P1.18-1,3,6. In other embodiments a strain may have been down-regulated for PorA expression e.g. in which the amount of PorA has been reduced by at least 20% (e.g. ≧30%, ≧40%, ≧50%, ≧60%, ≧70%, ≧80%, ≧90%, ≧95%, etc.), or even knocked out, relative to wild-type levels (e.g. relative to strain H44/76, as disclosed in reference 124).

In some embodiments a strain may hyper-express (relative to the corresponding wild-type strain) certain proteins. For instance, strains may hyper-express NspA, protein 287 [118], gHBP [114], TbpA and/or TbpB [119], Cu,Zn-superoxide dismutase [119], etc. As mentioned above, in some embodiments a meningococcus will hyper-express (relative to the corresponding wild-type strain) a polypeptide of the invention. Thus a gene encoding the polypeptide may be placed under the control of a promoter that leads to more expression than the wild-type strain's promoter, or the strain may be provided with a non-native polypeptide-coding sequence e.g. by integration into the chromosome or within a plasmid.

In some embodiments a strain may include one or more of the knockout and/or hyper-expression mutations disclosed in references 120 to 123. Preferred genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB [120]; (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/or TbpB [121]; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB [122]; and (d) CtrA, CtrB, CtrD, FrpB, OpA, OpC, PilC, PorB, SiaD, SynA, SynB, and/or SynC [123].

Where a mutant strain is used, in some embodiments it may have one or more, or all, of the following characteristics: (i) down-regulated or knocked-out LgtB and/or GalE to truncate the meningococcal LOS; (ii) up-regulated TbpA; (iii) up-regulated NhhA; (iv) up-regulated Omp85; (v) up-regulated LbpA; (vi) up-regulated NspA; (vii) knocked-out PorA; (viii) down-regulated or knocked-out FrpB; (ix) down-regulated or knocked-out Opa; (x) down-regulated or knocked-out Opc; (xii) deleted cps gene complex. A truncated LOS can be one that does not include a sialyl-lacto-N-neotetraose epitope e.g. it might be a galactose-deficient LOS. The LOS may have no a chain.

If LOS is present in a vesicle it is possible to treat the vesicle so as to link its LOS and protein components (“intra-bleb” conjugation [123]).

The invention may be used with mixtures of vesicles from different strains. For instance, reference 124 discloses vaccine comprising multivalent meningococcal vesicle compositions, comprising a first vesicle derived from a meningococcal strain with a serosubtype prevalent in a country of use, and a second vesicle derived from a strain that need not have a serosubtype prevent in a country of use. Reference 125 also discloses useful combinations of different vesicles. A combination of vesicles from strains in each of the L2 and L3 immunotypes may be used in some embodiments.

In some embodiments of the inventin, an immunogenic composition of the invention does not include any meningococcal outer membrane vesicles.

### Further Antigenic Components of Compositions of the Invention

The invention also provides a composition comprising a polypeptide or the invention and one or more of the following further antigens:
a saccharide antigen from N. meningitidis serogroup A, C, W135 and/or Y (preferably all four), such as the oligosaccharide disclosed in ref. 126 from serogroup C [see also ref. 127] or the oligosaccharides of ref. 128.
a saccharide antigen from Streptococcus pneumoniae [e.g. 129, 130, 131].
an antigen from hepatitis A virus, such as inactivated virus [e.g. 132, 133].
an antigen from hepatitis B virus, such as the surface and/or core antigens [e.g. 133, 134].
a diphtheria antigen, such as a diphtheria toxoid [e.g. chapter 3 of ref. 135] e.g. the CRM₁₉₇ mutant [e.g. 136].
a tetanus antigen, such as a tetanus toxoid [e.g. chapter 4 of ref. 135].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 137 & 138].
a saccharide antigen from Haemophilus influenzae B [e.g. 127].
polio antigen(s) [e.g. 139, 140] such as IPV.
measles, mumps and/or rubella antigens [e.g. chapters 9, 10 & 11 of ref. 135].
influenza antigen(s) [e.g. chapter 19 of ref. 135], such as the haemagglutinin and/or neuraminidase surface proteins.
an antigen from Moraxella catarrhalis [e.g. 141].
an protein antigen from Streptococcus agalactiae (group B streptococcus) [e.g. 142, 143].
a saccharide antigen from Streptococcus agalactiae (group B streptococcus).
an antigen from Streptococcus pyogenes (group A streptococcus) [e.g. 143, 144, 145].
an antigen from Staphylococcus aureus [e.g. 146].

The composition may comprise one or more of these further antigens.

Toxic protein antigens may be detoxified where necessary (e.g. detoxification of pertussis toxin by chemical and/or genetic means [138]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates include diphtheria toxin, tetanus toxin, the N. meningitidis outer membrane protein [147], synthetic peptides [148,149], heat shock proteins [150,151], pertussis proteins [152,153], protein D from H. influenzae [154], cytokines [155], lymphokines [155], streptococcal proteins, hormones [155], growth factors [155], toxin A or B from C. difficile [156], iron-uptake proteins [157], etc. A preferred carrier protein is the CRM197 diphtheria toxoid [158].

Antigens in the composition will typically be present at a concentration of at least 1 μg/ml each. In general, the concentration of any given antigen will be sufficient to elicit an immune response against that antigen.

As an alternative to using proteins antigens in the immunogenic compositions of the invention, nucleic acid (preferably DNA e.g. in the form of a plasmid) encoding the antigen may be used.

Antigens are preferably adsorbed to an aluminium salt.

### Screening Methods

The invention provides a process for determining whether a test compound binds to a polypeptide of the invention. If a test compound binds to a polypeptide of the invention and this binding inhibits the life cycle of the N. meningitidis bacterium, then the test compound can be used as an antibiotic or as a lead compound for the design of antibiotics. The process will typically comprise the steps of contacting a test compound with a polypeptide of the invention, and determining whether the test compound binds to said polypeptide. Preferred polypeptides of the invention for use in these processes are enzymes (e.g. tRNA synthetases), membrane transporters and ribosomal polypeptides. Suitable test compounds include polypeptides, polypeptides, carbohydrates, lipids, nucleic acids (e.g. DNA, RNA, and modified forms thereof), as well as small organic compounds (e.g. MW between 200 and 2000 Da). The test compounds may be provided individually, but will typically be part of a library (e.g. a combinatorial library). Methods for detecting a binding interaction include NMR, filter-binding assays, gel-retardation assays, displacement assays, surface plasmon resonance, reverse two-hybrid etc. A compound which binds to a polypeptide of the invention can be tested for antibiotic activity by contacting the compound with GBS bacteria and then monitoring for inhibition of growth.

The invention also provides a process for preparing an active compound, such as an antibiotic, comprising steps of: (i) providing a polypeptide of the invention; (ii) identifying a compound which binds to the polypeptide; and (iii) synthesising the identified compound, thus providing the active compound.

The invention also provides a compound identified using these methods.

Preferably, the process comprises the steps of: (a) contacting a polypeptide of the invention with one or more candidate compounds to give a mixture; (b) incubating the mixture to allow polypeptide and the candidate compound(s) to interact; and (c) assessing whether the candidate compound binds to the polypeptide or modulates its activity.

Once a candidate compound has been identified in vitro as a compound that binds to a polypeptide of the invention then it may be desirable to perform further experiments to confirm the in vivo function of the compound in inhibiting bacterial growth and/or survival. Thus the method comprise the further step of contacting the compound with a N. meningitidis bacterium and assessing its effect.

The polypeptide used in the screening process may be free in solution, affixed to a solid support, located on a cell surface or located intracellularly. Preferably, the binding of a candidate compound to the polypeptide is detected by means of a label directly or indirectly associated with the candidate compound. The label may be a fluorophore, radioisotope, or other detectable label.

### General

The invention provides a computer-readable medium (e.g. a floppy disk, a hard disk, a CD-ROM, a

DVD etc.) and/or a computer memory and/or a computer database containing one or more of the sequences in the sequence listing.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

The N-terminus residues in the amino acid sequences in the sequence listing are given as the amino acid encoded by the first codon in the corresponding nucleotide sequence. Where the first codon is not ATG, it will be understood that it will be translated as methionine when the codon is a start codon, but will be translated as the indicated non-Met amino acid when the sequence is downstream of the start codon e.g. as a fusion partner. The invention specifically discloses and encompasses each of the amino acid sequences of the sequence listing having a N-terminus methionine residue (e.g. a formyl-methionine residue) in place of any indicated non-Met N-terminus residue.

Alternative start codons can be used in biology. The amino acid sequences in the sequence listing are based on particular start codons, but downstream start codons may alternatively be used. Thus the invention specifically discloses and encompasses each of the amino acid sequences of the sequence listing (and variants thereof, as disclosed herein), starting at any methionine residue in the sequence that is downstream of the N-terminal residue shown in the sequence listing and excluding the upstream residues (e.g. a fragment of SEQ ID NO: 3 which starts at residue 53 or 57, rather than at residue 1; or a fragment of SEQ ID NO: 15955 which starts at residue 74, rather than at residue 1).

As indicated in the above text, nucleic acids and polypeptides of the invention may include sequences that:
(a) are identical (i.e. 100% identical) to the sequences disclosed in the sequence listing;
(b) share sequence identity with the sequences disclosed in the sequence listing;
(c) have 1, 2, 3, 4, 5, 6, 7, 8, 9 or 10 single nucleotide or amino acid alterations (deletions, insertions, substitutions), which may be at separate locations or may be contiguous, as compared to the sequences of (a) or (b); and
(d) when aligned with a particular sequence from the sequence listing using a pairwise alignment algorithm, a moving window of x monomers (amino acids or nucleotides) moving from start (N-terminus or 5′) to end (C-terminus of 3′), such that for an alignment that extends to p monomers (where p>x) there are p−x+1 such windows, each window has at least x·y identical aligned monomers, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x·y is is not an integer then it is rounded up to the nearest integer. The preferred pairwise alignment algorithm is the Needleman-Wunsch global alignment algorithm [159], using default parameters (e.g. with Gap opening penalty=10.0, and with Gap extension penalty=0.5, using the EBLOSUM62 scoring matrix). This algorithm is conveniently implemented in the needle tool in the EMBOSS package [160].

The nucleic acids and polypeptides of the invention may additionally have further sequences to the N-terminus/5′ and/or C-terminus/3′ of sequences (a) to (d); in some embodiments, however, they do not have any further sequences to the N-terminus/5′ and/or C-terminus/3′ of sequences (a) to (d).

The practice of the present invention will employ, unless otherwise indicated, conventional methods of chemistry, biochemistry, molecular biology, immunology and pharmacology, within the skill of the art. Such techniques are explained fully in the literature, e.g. references 24, 25 & 161-166, etc.

### MODES FOR CARRYING OUT THE INVENTION

18 different strains of meningococcus are selected. Details of their clonal complex and serological typing are as follows:

| Strain | Clonal complex | Serological typing |
|---|---|---|
| A | — | — |
| B | ST-8/A4 | B:2b:P1.21,16 |
| C | — | — |
| D | ST-32/ET-5 | B:4:P1.15 |
| E | ST-11/ET-37 | B:2a:P1.5 |
| F | ST-11/ET-37 | C:2a:P1.5,2 |
| G | ST-8/A4 | B:— |
| H | ST-269 | B:NT:P1.22,9 |
| I | ST-32/ET-5 | B:15:P1.7,16 |
| J | ST-41/44/Lin.3 | B:NT:P1.5,2 |
| K | ST-11/ET-37 | B:2a:P1.5,2 |
| L | ST-269 | B:— |
| M | ST-41/44/Lin.3 | B:4:P1.7,4 |
| N | ST-213 | B:1:P1.22,14 |
| O | ST-269 | B:NT:P1.19,15 |
| P | — | — |
| Q | ST-41/44/Lin.3 | B:4:P1.7-2.4 |
| R | ST-41/44/Lin.3 | B:— |

Genome sequencing of the 18 strains identifies 36178 encoded polypeptides. These are SEQ ID NOs: 1-36178 in the sequence listing. Their gene sequences encoding these 36175 polypeptides are SEQ ID NOs: 36179-72356. These SEQ IDs are from the 18 strains as follows:

| | Total | | |
|---|---|---|---|
| Strain | polypeptides | Polypeptide SEQ ID NOs | DNA SEQ ID NOs |
| A | 1925 | 1-1925 | 36179-38103 |
| B | 2050 | 1926-3975 | 38104-40153 |
| C | 1928 | 3976-5903 | 40154-42081 |
| D | 2032 | 5904-7935 | 42082-44113 |
| E | 2009 | 7936-9944 | 44114-46122 |
| F | 1983 | 9945-11927 | 46123-48105 |
| G | 1978 | 11928-13905 | 48106-50083 |
| H | 2089 | 13906-15993 & 36176 | 50084-52171 & 72354 |
| I | 2028 | 15994-18021 | 52172-54199 |
| J | 2025 | 18022-20046 | 54200-56224 |
| K | 2023 | 20047-22069 | 56225-58247 |
| L | 2042 | 22070-24111 | 58248-60289 |
| M | 1989 | 24112-26100 | 60290-62278 |
| N | 2025 | 26101-28125 | 62279-64303 |
| O | 2025 | 28126-30150 | 64304-66328 |
| P | 2071 | 30151-32221 | 66329-68399 |
| Q | 2018 | 32222-34237 & 36177-8 | 68400-70415 & 72355-6 |
| R | 1938 | 34238-36175 | 70416-72353 |

Properties and functions of these polypeptides are listed in Table 1.

### Lipoproteins

Of the 36175 coding sequences, the following 706 SEQ ID NOs are identified as lipoproteins: 38, 44, 49, 54, 115, 138, 281, 289, 295, 325, 470, 471, 519, 575, 618, 678, 715, 978, 998, 1063, 1209, 1365, 1366, 1435, 1506, 1583, 1620, 1684, 1686, 1717, 1752, 2086, 2101, 2269, 2320, 2332, 2341, 2347, 2369, 2379, 2455, 2497, 2701, 2835, 2862, 2944, 3067, 3142, 3171, 3172, 3235, 3241, 3245, 3342, 3384, 3392, 3426, 3453, 3484, 3502, 3575, 3664, 3666, 3732, 3791, 3954, 3971, 3990, 4071, 4178, 4289, 4372, 4770, 4788, 4864, 4932, 4936, 5134, 5345, 5430, 5524, 5561, 5617, 5658, 5675, 5681, 5796, 5809, 5925, 5953, 5968, 5982, 5985, 6061, 6070, 6103, 6212, 6216, 6236, 6253, 6419, 6425, 6457, 6511, 6541, 6705, 6767, 6776, 6806, 6839, 6874, 6931, 7029, 7034, 7051, 7184, 7209, 7214, 7230, 7295, 7351, 7435, 7463, 7490, 7528, 7579, 7625, 7632, 7725, 7731, 7736, 7807, 7808, 7923, 7994, 8060, 8149, 8191, 8193, 8247, 8295, 8339, 8361, 8490, 8509, 8558, 8651, 8719, 8750, 8762, 8788, 8853, 8879, 8885, 8951, 9033, 9034, 9060, 9103, 9189, 9194, 9213, 9321, 9378, 9400, 9420, 9468, 9472, 9484, 9686, 9728, 9767, 9770, 9850, 9950, 9969, 10015, 10025, 10049, 10052, 10266, 10360, 10369, 10405, 10441, 10511, 10527, 10532, 10673, 10862, 10932, 10980, 10983, 11061, 11081, 11126, 11159, 11163, 11186, 11362, 11465, 11488, 11496, 11513, 11526, 11533, 11555, 11576, 11591, 11674, 11731, 11747, 11767, 11948, 12005, 12026, 12056, 12082, 12087, 12101, 12120, 12122, 12238, 12301, 12349, 12380, 12390, 12424, 12433, 12556, 12605, 12613, 12680, 12753, 12793, 12880, 12886, 12899, 12906, 13025, 13072, 13419, 13520, 13592, 13628, 13639, 13658, 13668, 13753, 13816, 13984, 14013, 14071, 14189, 14212, 14216, 14222, 14228, 14317, 14349, 14355, 14379, 14528, 14541, 14598, 14632, 14633, 14719, 14798, 14832, 14897, 15038, 15057, 15088, 15153, 15192, 15205, 15216, 15222, 15258, 15268, 15285, 15329, 15360, 15402, 15479, 15514, 15533, 15639, 15717, 15773, 15780, 15785, 15858, 15859, 15867, 16172, 16181, 16182, 16206, 16218, 16219, 16233, 16249, 16383, 16561, 16604, 16616, 16630, 16643, 16687, 16740, 16761, 16793, 16831, 16836, 16891, 16922, 16943, 16951, 17002, 17083, 17095, 17101, 17131, 17164, 17297, 17313, 17357, 17358, 17399, 17483, 17515, 17566, 17738, 17792, 17862, 17869, 18014, 18060, 18075, 18117, 18179, 18188, 18216, 18219, 18281, 18299, 18339, 18368, 18411, 18426, 18456, 18505, 18539, 18541, 18559, 18564, 18791, 18843, 19074, 19158, 19202, 19298, 19318, 19336, 19464, 19489, 19550, 19590, 19593, 19639, 19640, 19702, 19717, 19746, 19792, 19875, 19880, 20058, 20084, 20115, 20160, 20188, 20193, 20220, 20225, 20228, 20288, 20350, 20386, 20440, 20478, 20510, 20533, 20595, 20660, 20683, 20756, 20844, 20855, 20935, 20949, 20984, 20997, 21032, 21063, 21090, 21096, 21104, 21338, 21376, 21464, 21492, 21530, 21623, 21628, 21726, 21751, 21905, 21969, 22108, 22111, 22112, 22125, 22148, 22194, 22269, 22322, 22445, 22494, 22505, 22577, 22666, 22787, 22802, 22858, 22860, 22894, 22897, 22930, 23178, 23262, 23281, 23290, 23366, 23390, 23411, 23543, 23561, 23569, 23607, 23609, 23792, 23800, 23868, 23929, 23953, 23974, 24032, 24033, 24120, 24257, 24276, 24362, 24401, 24438, 24492, 24525, 24539, 24586, 24697, 24702, 24709, 24732, 24746, 24783, 24795, 24820, 24875, 24886, 24922, 24953, 24956, 24980, 25088, 25104, 25250, 25304, 25306, 25324, 25448, 25489, 25603, 25606, 25613, 25634, 25652, 25754, 25954, 26096, 26207, 26257, 26263, 26292, 26349, 26445, 26511, 26544, 26774, 26809, 26819, 26820, 26829, 26843, 26941, 26962, 26978, 26994, 27110, 27211, 27229, 27291, 27434, 27473, 27514, 27518, 27541, 27636, 27645, 27675, 27698, 27713, 27719, 27756, 27887, 27963, 28025, 28026, 28063, 28228, 28255, 28280, 28328, 28355, 28386, 28470, 28472, 28487, 28515, 28536, 28611, 28626, 28671, 28749, 28751, 28763, 28795, 28836, 28872, 28973, 28986, 29007, 29035, 29102, 29167, 29218, 29223, 29416, 29465, 29479, 29506, 29546, 29629, 29654, 29684, 29737, 29814, 29819, 29851, 29857, 29866, 29907, 29993, 29994, 30066, 30266, 30311, 30326, 30471, 30474, 30528, 30614, 30630, 30647, 30802, 30805, 30812, 30826, 30879, 30982, 31069, 31073, 31093, 31105, 31122, 31193, 31231, 31236, 31290, 31480, 31490, 31498, 31499, 31578, 31603, 31611, 31693, 31719, 31758, 31792, 31819, 31828, 31890, 31924, 32107, 32322, 32417, 32525, 32583, 32616, 32709, 32761, 32762, 32799, 32857, 32882, 32892, 32915, 32974, 33419, 33458, 33479, 33521, 33529, 33533, 33549, 33555, 33602, 33638, 33642, 33726, 33735, 33749, 33755, 33790, 33798, 33820, 33825, 33893, 33920, 33966, 33993, 34003, 34009, 34035, 34098, 34156, 34232, 34259, 34261, 34269, 34299, 34367, 34403, 34459, 34643, 34753, 34801, 34867, 34885, 34931, 34941, 35003, 35023, 35043, 35101, 35132, 35163, 35209, 35324, 35372, 35397, 35561, 35614, 35636, 35654, 35691, 35692, 35710, 35781, 35868, 35934, 36068, 36075, and 36153.

Lipoproteins are surface-exposed and, as such, they represent accessible immunological targets e.g. for diagnostic and for immunisation purposes. Moreover, it has been found in B. burgdorferi [167] that OspA protein is immunogenic in a lipidated form but is non-immunogenic in a non-lipidated form, and the authors concluded that post-translational lipid attachment is a critical determinant of OspA immunogenicity.

Lipoproteins of the invention are ideally provided in mature form e.g. without their leader peptides and with a N-terminus lipidated cysteine residue. The N-terminus residue may have an attached palmitoyl group e.g. to provide a tripalmitoyl-S-glyceryl-cysteine amino acid.

### Outer Membrane

As N. meningitidis is a Gram-negative bacterium, its cell wall includes an outer membrane. Of the 36175 sequences, the following 273 SEQ ID NOs are identified as being located in this outer membrane: 246, 519, 624, 824, 918, 949, 964, 987, 1078, 1207, 1332, 1366, 1427, 1534, 1599, 1694, 1991, 2101, 2276, 2379, 2465, 2950, 3067, 3280, 3290, 3363, 3542, 3552, 3712, 3944, 3957, 3960, 4004, 4319, 4722, 4783, 5318, 5345, 5364, 5430, 5436, 5482, 5590, 5593, 5617, 5650, 5651, 5729, 5787, 6103, 6280, 6760, 6835, 6836, 6915, 7118, 7211, 7542, 7726, 7731, 7930, 7932, 7994, 8134, 8157, 8324, 8667, 8879, 9211, 9415, 9549, 9699, 9798, 9919, 9929, 9930, 9977, 10126, 10165, 10279, 10288, 10302, 10367, 10368, 10454, 10456, 10504, 10516, 10527, 10563, 10664, 10673, 10805, 10928, 11019, 11251, 11410, 11488, 11576, 11666, 11677, 11702, 11781, 11824, 11862, 11918, 12007, 12088, 12147, 12602, 12605, 12906, 13076, 13353, 13811, 13843, 13903, 13904, 13905, 14169, 14349, 15111, 15217, 15232, 15316, 15337, 15445, 15485, 15575, 15748, 15780, 15804, 15834, 15954, 15955, 16075, 16262, 16470, 16633, 16687, 16975, 17419, 17478, 17515, 17768, 17876, 18019, 18020, 18037, 18146, 18406, 18537, 18756, 19074, 19187, 19382, 19470, 19822, 19880, 19893, 20084, 20656, 20742, 21090, 21242, 21526, 21536, 21641, 21695, 21786, 21830, 21929, 21949, 22060, 22062, 22224, 22426, 22509, 22608, 22836, 23015, 23262, 23373, 23417, 23569, 23904, 24028, 24109, 24111, 24610, 24618, 24699, 24702, 24737, 24858, 25090, 25324, 25726, 25961, 26045, 26098, 26100, 26164, 26191, 26443, 26459, 26994, 27518, 27585, 27660, 27701, 27797, 27973, 28051, 28122, 28124, 28432, 28705, 29007, 29241, 29453, 29499, 29518, 29546, 29784, 29805, 30036, 30067, 30094, 30149, 30150, 30227, 30375, 30614, 30873, 30930, 31603, 31950, 32081, 32103, 32112, 32114, 32116, 32195, 32254, 32457, 32774, 33118, 33466, 33480, 33521, 33541, 33642, 33683, 33938, 34105, 34236, 34237, 34328, 34330, 34359, 34834, 34846, 34949, 35132, 35692, 35745, 36005, 36018, 36144, 36153, 36168, and 36169.

In particular, the following 55 SEQ ID NOs: are identified as adhesins: 213, 397, 941, 1561, 1659, 1678, 1902, 2841, 3936, 3959, 4030, 4251, 4305, 4711, 4875, 5023, 5064, 5288, 5457, 5605, 5766, 6744, 7226, 7547, 7575, 7577, 9875, 10075, 10631, 10722, 11221, 11278, 11432, 11476, 11485, 11501, 11733, 11760, 12893, 14878, 15050, 18027, 18043, 18790, 19465, 20355, 20436, 20500, 20900, 21016, 28054, 30845, 32131, 32164, 32207.

The following 52 SEQ ID NOs are PilC-related: 3282, 3706, 6675, 7553, 9059, 9076, 10967, 12832, 14200, 14742, 17485, 17511, 18269, 18407, 20847, 21435, 22308, 22525, 25137, 25850, 27453, 27581, 28186, 30080, 31206, 34162, 34166, 34583, 36148, 39457, 39881, 42850, 43728, 45234, 45251, 47142, 49007, 50375, 50917, 53660, 53686, 54444, 54582, 57022, 57610, 58483, 58700, 61312, 62025, 63628, 63756, 64361. PilC is a known cell-surface protein.

Outer membrane proteins (OMPs) are surface-exposed and, as such, they represent accessible immunological targets e.g. for diagnostic and for immunisation purposes. OMPs are often invasins, adhesins, etc. which, if blocked, offers a means of preventing bacterial infection.

### Periplasm

As N. meningitidis is a Gram-negative bacterium, it has a periplasm between its cell cytoplasmic membrane and its outer membrane. Of the 36175 sequences, the following 364 SEQ ID NOs: are identified as being located in the periplasm: 12, 114, 172, 184, 242, 254, 314, 388, 483, 499, 503, 548, 603, 620, 649, 671, 683, 687, 691, 718, 722, 742, 745, 747, 783, 807, 810, 812, 903, 911, 1014, 1043, 1051, 1133, 1170, 1182, 1186, 1195, 1199, 1251, 1287, 1391, 1394, 1438, 1527, 1549, 1580, 1594, 1629, 1646, 1673, 1828, 2547, 2597, 2670, 2789, 2865, 2873, 2947, 3096, 3131, 3510, 3596, 3729, 3975, 4004, 4056, 4239, 4332, 4358, 4424, 4455, 4514, 4544, 4644, 4659, 4717, 4748, 4794, 4892, 4895, 4900, 4967, 5094, 5114, 5159, 5169, 5197, 5530, 5760, 5772, 5946, 5994, 6290, 6370, 6435, 6584, 6637, 6826, 6886, 6979, 7003, 7131, 7169, 7393, 7425, 7439, 7501, 7631, 7659, 7974, 8694, 8772, 8826, 8834, 8986, 8989, 8994, 9080, 9169, 9422, 9528, 9561, 9665, 9967, 10031, 10050, 10064, 10070, 10093, 10141, 10162, 10174, 10207, 10215, 10236, 10260, 10265, 10277, 10378, 10392, 10401, 10408, 10410, 10471, 10496, 10526, 10535, 10551, 10558, 10598, 10662, 10664, 10679, 10687, 10700, 10704, 10761, 10784, 10797, 10813, 10854, 10931, 10961, 10982, 11107, 11138, 11146, 11161, 11218, 11297, 11347, 11376, 11396, 11422, 11424, 11455, 11510, 11548, 11613, 11670, 11700, 11764, 11818, 11820, 11847, 12042, 12127, 12397, 12519, 12755, 12777, 12895, 13178, 13366, 13421, 13666, 13611, 13615, 13647, 14058, 14097, 14110, 14120, 14196, 14553, 14956, 15020, 15252, 15272, 15308, 15452, 15507, 15890, 15950, 16078, 16269, 16295, 16460, 16481, 16896, 16958, 17136, 17147, 17270, 17570, 17672, 17674, 17687, 18061, 18069, 18086, 18088, 18108, 18116, 18128, 18153, 18159, 18442, 18916, 19104, 19111, 19154, 19231, 19317, 19403, 19737, 19829, 19976, 20179, 20237, 20406, 20415, 20441, 20444, 20876, 20901, 20946, 20950, 21030, 21350, 21503, 21618, 21640, 21987, 22064, 22229, 22271, 22655, 22672, 22703, 22715, 23208, 23392, 23422, 23503, 23586, 23726, 23731, 23890, 24180, 24344, 24347, 24493, 24683, 24949, 25010, 25360, 25560, 25568, 25725, 25795, 25853, 25906, 26146, 26317, 26352, 26381, 26515, 26815, 26972, 27311, 27392, 27475, 27528, 27617, 27761, 27827, 28283, 28406, 28645, 28715, 28803, 29081, 29128, 29147, 29188, 29381, 29620, 29877, 29939, 30088, 30202, 30466, 30509, 30694, 30700, 30748, 31278, 31283, 31292, 31331, 31336, 31463, 31627, 31815, 32247, 32284, 32402, 32474, 32482, 33049, 33437, 33456, 33493, 33582, 33622, 33892, 33975, 34171, 34396, 34414, 34441, 34547, 34570, 34683, 34965, 34980, 35314, 35381, 35657, 35836, 35939, and 36065.

Periplasmic proteins represent useful immunological targets e.g. for diagnostic and for immunisation purposes.

### Inner Membrane

As N. meningitidis is a Gram-negative bacterium, it has an inner membrane. Of the 376175 coding sequences, the following 98 are identified as being located in the inner membrane: 98, 362, 469, 565, 787, 886, 1269, 1397, 1731, 1739, 1782, 1800, 2984, 3410, 3993, 4162, 4365, 4762, 5131, 5461, 7258, 8630, 8829, 8907, 10039, 10099, 10113, 10117, 10334, 10344, 10403, 10434, 10444, 10455, 10473, 10486, 10505, 10528, 10538, 10648, 10711, 10730, 10899, 10954, 11045, 11048, 11068, 11072, 11097, 11102, 11122, 11127, 11131, 11143, 11196, 11226, 11231, 11272, 11405, 11502, 11579, 11609, 11630, 11649, 11685, 11699, 11730, 11775, 11776, 11777, 11779, 11805, 11815, 11915, 11932, 12170, 13986, 14181, 16901, 17942, 19141, 20171, 21515, 22278, 24829, 25705, 26036, 27155, 27323, 28033, 28772, 30249, 31057, 32014, 32289, 35227, 36076, and 36085. Inner membrane proteins represent useful immunological targets e.g. for diagnostic and for immunisation purposes.

### The “Universal Vaccine” of Reference 3

The “universal vaccine” of reference 3 includes five different meningococcal antigens. By standard genome numbering from the MC58 strain these are NMB1030, NMB1870, NMB1994, NMB2091 and NMB2132. The sequence listing includes polymorphic forms of these five antigens:

| NMB | Polymorphs | SEQ ID NOs |
|---|---|---|
| 1030 | 23 | 72357-72379 |
| 1870 | 323 | 72380-72702 |
| 1994 | 74 | 72703-72776 |
| 2091 | 16 | 72777-72792 |
| 2132 | 198 | 72793-72990 |

These polymorphs can be used to replace the sequences which were used in reference 3.

It will be understood that the invention has been described by way of example only and modifications may be made whilst remaining within the scope and spirit of the invention.

### REFERENCES (The Contents of which are Hereby Incorporated by Reference)

[1] Tettelin et al. (2000) Science 287:1809-15.
[2] Parkhill et al. (2000) Nature 404:502-6.
[3] Giuliani et al. (2006) PNAS USA 10834-9.
[4] Geysen et al. (1984) PNAS USA 81:3998-4002.
[5] Carter (1994) Methods Mol Biol 36:207-23.
[6] Jameson, B A et al. 1988, CABIOS 4(1):181-186.
[7] Raddrizzani & Hammer (2000) Brief Bioinform 1(2):179-89.
[8] De Lalla et al. (1999) J. Immunol. 163:1725-29.
[9] Brusic et al. (1998) Bioinformatics 14(2):121-30
[10] Meister et al. (1995) Vaccine 13(6):581-91.
[11] Roberts et al. (1996) AIDS Res Hum Retroviruses 12(7):593-610.
[12] Maksyutov & Zagrebelnaya (1993) Comput Appl Biosci 9(3):291-7.
[13] Feller & de la Cruz (1991) Nature 349(6311):720-1.
[14] Hopp (1993) Peptide Research 6:183-190.
[15] Welling et al. (1985) FEBS Lett. 188:215-218.
[16] Davenport et al. (1995) Immunogenetics 42:392-297.
[17] Bodanszky (1993) Principles of Peptide Synthesis (ISBN: 0387564314).
[18] Fields et al. (1997) Meth Enzymol 289: Solid-Phase Peptide Synthesis. ISBN: 0121821900.
[19] Chan & White (2000) Fmoc Solid Phase Peptide Synthesis. ISBN: 0199637245.
[20] Kullmann (1987) Enzymatic Peptide Synthesis. ISBN: 0849368413.
[21] Ibba (1996) Biotechnol Genet Eng Rev 13:197-216.
[22] Breedveld (2000) Lancet 355(9205):735-740.
[23] Gorman & Clark (1990) Semin. Immunol. 2:457-466.
[24] Sambrook et al. (1989) Molecular Cloning: A Laboratory Manual.
[25] Short protocols in molecular biology (4th ed, 1999) Ausubel et al. eds. ISBN 0-471-32938-X.
[26] U.S. Pat. No. 5,707,829
[27] Current Protocols in Molecular Biology (F. M. Ausubel et al. eds., 1987) Supplement 30.
[28] EP-B-0509612.
[29] EP-B-0505012.
[30] Drakopoulou et al. (2008) FEMS Immunol Med Microbiol 53:178-82.
[31] Fernández-Rodríguez et al. (2008) Diagn Microbiol Infect Dis. 60:339-46.
[32] Tang et al. (1997) Clin. Chem. 43:2021-2038.
[33] Vaccine Design . . . (1995) eds. Powell & Newman. ISBN: 030644867X. Plenum.
[34] WO00/23105.
[35] WO90/14837.
[36] U.S. Pat. No. 5,057,540.
[37] WO96/33739.
[38] EP-A-0109942.
[39] WO96/11711.
[40] WO00/07621.
[41] Barr et al. (1998) Advanced Drug Delivery Reviews 32:247-271.
[42] Sjolanderet et al. (1998) Advanced Drug Delivery Reviews 32:321-338.
[43] Niikura et al. (2002) Virology 293:273-280.
[44] Lenz et al. (2001) J Immunol 166:5346-5355.
[45] Pinto et al. (2003) J Infect Dis 188:327-338.
[46] Gerber et al. (2001) Virol 75:4752-4760.
[47] WO03/024480
[48] WO03/024481
[49] Gluck et al. (2002) Vaccine 20:B10-B16.
[50] EP-A-0689454.
[51] Johnson et al. (1999) Bioorg Med Chem Lett 9:2273-2278.
[52] Evans et al. (2003) Expert Rev Vaccines 2:219-229.
[53] Meraldi et al. (2003) Vaccine 21:2485-2491.
[54] Pajak et al. (2003) Vaccine 21:836-842.
[55] Kandimalla et al. (2003) Nucleic Acids Research 31:2393-2400.
[56] WO02/26757.
[57] WO99/62923.
[58] Krieg (2003) Nature Medicine 9:831-835.
[59] McCluskie et al. (2002) FEMS Immunology and Medical Microbiology 32:179-185.
[60] WO98/40100.
[61] U.S. Pat. No. 6,207,646.
[62] U.S. Pat. No. 6,239,116.
[63] U.S. Pat. No. 6,429,199.
[64] Kandimalla et al. (2003) Biochemical Society Transactions 31 (part 3):654-658.
[65] Blackwell et al. (2003) J Immunol 170:4061-4068.
[66] Krieg (2002) Trends Immunol 23:64-65.
[67] WO01/95935.
[68] Kandimalla et al. (2003) BBRC 306:948-953.
[69] Bhagat et al. (2003) BBRC 300:853-861.
[70] WO03/035836.
[71] WO95/17211.
[72] WO98/42375.
[73] Beignon et al. (2002) Infect Immun 70:3012-3019.
[74] Pizza et al. (2001) Vaccine 19:2534-2541.
[75] Pizza et al. (2000) Int J Med Microbiol 290:455-461.
[76] Scharton-Kersten et al. (2000) Infect Immun 68:5306-5313.
[77] Ryan et al. (1999) Infect Immun 67:6270-6280.
[78] Partidos et al. (1999) Immunol Lett 67:209-216.
[79] Peppoloni et al. (2003) Expert Rev Vaccines 2:285-293.
[80] Pine et al. (2002) J Control Release 85:263-270.
[81] Domenighini et al. (1995) Mol Microbiol 15:1165-1167.
[82] WO99/40936.
[83] WO99/44636.
[84] Singh et al] (2001) J Cont Release 70:267-276.
[85] WO99/27960.
[86] U.S. Pat. No. 6,090,406
[87] U.S. Pat. No. 5,916,588
[88] EP-A-0626169.
[89] WO99/52549.
[90] WO01/21207.
[91] WO01/21152.
[92] Andrianov et al. (1998) Biomaterials 19:109-115.
[93] Payne et al. (1998) Adv Drug Delivery Review 31:185-196.
[94] Stanley (2002) Clin Exp Dermatol 27:571-577.
[95] Jones (2003) Curr Opin Investig Drugs 4:214-218.
[96] WO99/11241.
[97] WO94/00153.
[98] WO98/57659.
[99] European patent applications 0835318, 0735898 and 0761231.
[100] WO03/009869.
[101] Almeida & Alpar (1996) J. Drug Targeting 3:455-467.
[102] Agarwal & Mishra (1999) Indian J Exp Biol 37:6-16.
[103] WO02/09643.
[104] Katial et al. (2002) Infect. Immun. 70:702-707.
[105] U.S. Pat. No. 6,180,111.
[106] WO01/34642.
[107] WO2004/019977.
[108] European patent 0011243.
[109] Fredriksen et al. (1991) NIPH Ann. 14(2):67-80.
[110] WO01/91788.
[111] WO2005/004908.
[112] Maiden et al. (1998) PNAS USA 95:3140-3145.
[113] WO98/56901.
[114] WO2006/081259.
[115] Claassen et al. (1996) 14(10):1001-8.
[116] WO99/10497.
[117] Steeghs et al. (2001) The EMBO Journal 20:6937-6945.
[118] WO01/52885.
[119] WO00/25811.
[120] WO01/09350.
[121] WO02/09746.
[122] WO02/062378.
[123] WO2004/014417.
[124] WO03/105890.
[125] WO2006/024946
[126] Costantino et al. (1992) Vaccine 10:691-698.
[127] Costantino et al. (1999) Vaccine 17:1251-1263.
[128] WO03/007985.
[129] Watson (2000) Pediatr Infect Dis J 19:331-332.
[130] Rubin (2000) Pediatr Clin North Am 47:269-285, v.
[131] Jedrzejas (2001) Microbiol Mol Biol Rev 65:187-207.
[132] Bell (2000) Pediatr Infect Dis J 19:1187-1188.
[133] Iwarson (1995) APMIS 103:321-326.
[134] Gerlich et al. (1990) Vaccine 8 Suppl:S63-68 & 79-80.
[135] Vaccines (1988) eds. Plotkin & Mortimer. ISBN 0-7216-1946-0.
[136] Del Guidice et al. (1998) Molecular Aspects of Medicine 19:1-70.
[137] Gustafsson et al. (1996) N. Engl. J. Med. 334:349-355.
[138] Rappuoli et al. (1991)TIBTECH 9:232-238.
[139] Sutter et al. (2000) Pediatr Clin North Am 47:287-308.
[140] Zimmerman & Spann (1999) Am Fam Physician 59:113-118, 125-126.
[141] McMichael (2000) Vaccine 19 Suppl 1:S101-107.
[142] Schuchat (1999) Lancet 353(9146):51-6.
[143] WO02/34771.
[144] Dale (1999) Infect Dis Clin North Am 13:227-43, viii.
[145] Ferretti et al. (2001) PNAS USA 98: 4658-4663.
[146] Kuroda et al. (2001) Lancet 357(9264):1225-1240; see also pages 1218-1219.
[147] EP-A-0372501
[148] EP-A-0378881
[149] EP-A-0427347
[150] WO93/17712
[151] WO94/03208
[152] WO98/58668
[153] EP-A-0471177
[154] WO00/56360
[155] WO91/01146
[156] WO00/61761
[157] WO01/72337
[158] Research Disclosure, 453077 (Jan 2002)
[159] Needleman & Wunsch (1970) J. Mol. Biol. 48, 443-453.
[160] Rice et al. (2000) Trends Genet 16:276-277.
[161] Gennaro (2000) Remington: The Science and Practice of Pharmacy. 20th edition, ISBN: 0683306472.
[162] Methods In Enzymology (S. Colowick and N. Kaplan, eds., Academic Press, Inc.)
[163] Handbook of Experimental Immunology, Vols. I-IV (D. M. Weir and C. C. Blackwell, eds., 1986, Blackwell Scientific Publications)
[164] Handbook of Surface and Colloidal Chemistry (Birdi, K. S. ed., CRC Press, 1997)
[165] Molecular Biology Techniques: An Intensive Laboratory Course, (Ream et al., eds., 1998, Academic Press)
[166] PCR Introduction to Biotechniques Series 2nd ed. 1997 (Newton & Graham, Springer Verlag).
[167] Erdile et al. (1993) Infect Immun 61:81-90.

TABLE 2
a list of SEQ ID NOs of interest

12, 38, 44, 49, 54, 98, 114, 115, 138, 172, 184, 213, 242, 246, 254, 281, 289, 295, 314, 325, 362,
388, 397, 469, 470, 471, 483, 499, 503, 519, 519, 548, 565, 575, 603, 618, 620, 624, 649, 671, 678,
683, 687, 691, 715, 718, 722, 742, 745, 747, 783, 787, 807, 810, 812, 824, 886, 903, 911, 918, 941,
949, 964, 978, 987, 998, 1014, 1043, 1051, 1063, 1078, 1133, 1170, 1182, 1186, 1195, 1199, 1207,
1209, 1251, 1269, 1287, 1332, 1365, 1366, 1366, 1391, 1394, 1397, 1427, 1435, 1438, 1506, 1527,
1534, 1549, 1561, 1580, 1583, 1594, 1599, 1620, 1629, 1646, 1659, 1673, 1678, 1684, 1686, 1694,
1717, 1731, 1739, 1752, 1782, 1800, 1828, 1902, 1991, 2086, 2101, 2101, 2269, 2276, 2320, 2332,
2341, 2347, 2369, 2379, 2379, 2455, 2465, 2497, 2547, 2597, 2670, 2701, 2789, 2835, 2841, 2862,
2865, 2873, 2944, 2947, 2950, 2984, 3067, 3067, 3096, 3131, 3142, 3171, 3172, 3235, 3241, 3245,
3280, 3290, 3342, 3363, 3384, 3392, 3410, 3426, 3453, 3484, 3502, 3510, 3542, 3552, 3575, 3596,
3664, 3666, 3712, 3729, 3732, 3791, 3936, 3944, 3954, 3957, 3959, 3960, 3971, 3975, 3990, 3993,
4004, 4004, 4030, 4056, 4071, 4162, 4178, 4239, 4251, 4289, 4305, 4319, 4332, 4358, 4365, 4372,
4424, 4455, 4514, 4544, 4644, 4659, 4711, 4717, 4722, 4748, 4762, 4770, 4783, 4788, 4794, 4864,
4875, 4892, 4895, 4900, 4932, 4936, 4967, 5023, 5064, 5094, 5114, 5131, 5134, 5159, 5169, 5197,
5288, 5318, 5345, 5345, 5364, 5430, 5430, 5436, 5457, 5461, 5482, 5524, 5530, 5561, 5590, 5593,
5605, 5617, 5617, 5650, 5651, 5658, 5675, 5681, 5729, 5760, 5766, 5772, 5787, 5796, 5809, 5925,
5946, 5953, 5968, 5982, 5985, 5994, 6061, 6070, 6103, 6103, 6212, 6216, 6236, 6253, 6280, 6290,
6370, 6419, 6425, 6435, 6457, 6511, 6541, 6584, 6637, 6705, 6744, 6760, 6767, 6776, 6806, 6826,
6835, 6836, 6839, 6874, 6886, 6915, 6931, 6979, 7003, 7029, 7034, 7051, 7118, 7131, 7169, 7184,
7209, 7211, 7214, 7226, 7230, 7258, 7295, 7351, 7393, 7425, 7435, 7439, 7463, 7490, 7501, 7528,
7542, 7547, 7575, 7577, 7579, 7625, 7631, 7632, 7659, 7725, 7726, 7731, 7731, 7736, 7807, 7808,
7923, 7930, 7932, 7974, 7994, 7994, 8060, 8134, 8149, 8157, 8191, 8193, 8247, 8295, 8324, 8339,
8361, 8490, 8509, 8558, 8630, 8651, 8667, 8694, 8719, 8750, 8762, 8772, 8788, 8826, 8829, 8834,
8853, 8879, 8879, 8885, 8907, 8951, 8986, 8989, 8994, 9033, 9034, 9060, 9080, 9103, 9169, 9189,
9194, 9211, 9213, 9321, 9378, 9400, 9415, 9420, 9422, 9468, 9472, 9484, 9528, 9549, 9561, 9665,
9686, 9699, 9728, 9767, 9770, 9798, 9850, 9875, 9919, 9929, 9930, 9950, 9967, 9969, 9977, 10015,
10025, 10031, 10039, 10049, 10050, 10052, 10064, 10070, 10075, 10093, 10099, 10113, 10117,
10126, 10141, 10162, 10165, 10174, 10207, 10215, 10236, 10260, 10265, 10266, 10277, 10279,
10288, 10302, 10334, 10344, 10360, 10367, 10368, 10369, 10378, 10392, 10401, 10403, 10405,
10408, 10410, 10434, 10441, 10444, 10454, 10455, 10456, 10471, 10473, 10486, 10496, 10504,
10505, 10511, 10516, 10526, 10527, 10527, 10528, 10532, 10535, 10538, 10551, 10558, 10563,
10598, 10631, 10648, 10662, 10664, 10664, 10673, 10673, 10679, 10687, 10700, 10704, 10711,
10722, 10730, 10761, 10784, 10797, 10805, 10813, 10854, 10862, 10899, 10928, 10931, 10932,
10954, 10961, 10980, 10982, 10983, 11019, 11045, 11048, 11061, 11068, 11072, 11081, 11097,
11102, 11107, 11122, 11126, 11127, 11131, 11138, 11143, 11146, 11159, 11161, 11163, 11186,
11196, 11218, 11221, 11226, 11231, 11251, 11272, 11278, 11297, 11347, 11362, 11376, 11396,
11405, 11410, 11422, 11424, 11432, 11455, 11465, 11476, 11485, 11488, 11488, 11496, 11501,
11502, 11510, 11513, 11526, 11533, 11548, 11555, 11576, 11576, 11579, 11591, 11609, 11613,
11630, 11649, 11666, 11670, 11674, 11677, 11685, 11699, 11700, 11702, 11730, 11731, 11733,
11747, 11760, 11764, 11767, 11775, 11776, 11777, 11779, 11781, 11805, 11815, 11818, 11820,
11824, 11847, 11862, 11915, 11918, 11932, 11948, 12005, 12007, 12026, 12042, 12056, 12082,
12087, 12088, 12101, 12120, 12122, 12127, 12147, 12170, 12238, 12301, 12349, 12380, 12390,
12397, 12424, 12433, 12519, 12556, 12602, 12605, 12605, 12613, 12680, 12753, 12755, 12777,
12793, 12880, 12886, 12893, 12895, 12899, 12906, 12906, 13025, 13072, 13076, 13178, 13353,
13366, 13419, 13421, 13520, 13592, 13606, 13611, 13615, 13628, 13639, 13647, 13658, 13668,
13753, 13811, 13816, 13843, 13903, 13904, 13905, 13984, 13986, 14013, 14058, 14071, 14097,
14110, 14120, 14169, 14181, 14189, 14196, 14212, 14216, 14222, 14228, 14317, 14349, 14349,
14355, 14379, 14528, 14541, 14553, 14598, 14632, 14633, 14719, 14798, 14832, 14878, 14897,
14956, 15020, 15038, 15050, 15057, 15088, 15111, 15153, 15192, 15205, 15216, 15217, 15222,
15232, 15252, 15258, 15268, 15272, 15285, 15308, 15316, 15329, 15337, 15360, 15402, 15445,
15452, 15479, 15485, 15507, 15514, 15533, 15575, 15639, 15717, 15748, 15773, 15780, 15780,
15785, 15804, 15834, 15858, 15859, 15867, 15890, 15950, 15954, 15955, 16075, 16078, 16172,
16181, 16182, 16206, 16218, 16219, 16233, 16249, 16262, 16269, 16295, 16383, 16460, 16470,
16481, 16561, 16604, 16616, 16630, 16633, 16643, 16687, 16687, 16740, 16761, 16793, 16831,
16836, 16891, 16896, 16901, 16922, 16943, 16951, 16958, 16975, 17002, 17083, 17095, 17101,
17131, 17136, 17147, 17164, 17270, 17297, 17313, 17357, 17358, 17399, 17419, 17478, 17483,
17515, 17515, 17566, 17570, 17672, 17674, 17687, 17738, 17768, 17792, 17862, 17869, 17876,
17942, 18014, 18019, 18020, 18027, 18037, 18043, 18060, 18061, 18069, 18075, 18086, 18088,
18108, 18116, 18117, 18128, 18146, 18153, 18159, 18179, 18188, 18216, 18219, 18281, 18299,
18339, 18368, 18406, 18411, 18426, 18442, 18456, 18505, 18537, 18539, 18541, 18559, 18564,
18756, 18790, 18791, 18843, 18916, 19074, 19074, 19104, 19111, 19141, 19154, 19158, 19187,
19202, 19231, 19298, 19317, 19318, 19336, 19382, 19403, 19464, 19465, 19470, 19489, 19550,
19590, 19593, 19639, 19640, 19702, 19717, 19737, 19746, 19792, 19822, 19829, 19875, 19880,
19880, 19893, 19976, 20058, 20084, 20084, 20115, 20160, 20171, 20179, 20188, 20193, 20220,
20225, 20228, 20237, 20288, 20350, 20355, 20386, 20406, 20415, 20436, 20440, 20441, 20444,
20478, 20500, 20510, 20533, 20595, 20656, 20660, 20683, 20742, 20756, 20844, 20855, 20876,
20900, 20901, 20935, 20946, 20949, 20950, 20984, 20997, 21016, 21030, 21032, 21063, 21090,
21090, 21096, 21104, 21242, 21338, 21350, 21376, 21464, 21492, 21503, 21515, 21526, 21530,
21536, 21618, 21623, 21628, 21640, 21641, 21695, 21726, 21751, 21786, 21830, 21905, 21929,
21949, 21969, 21987, 22060, 22062, 22064, 22108, 22111, 22112, 22125, 22148, 22194, 22224,
22229, 22269, 22271, 22278, 22322, 22426, 22445, 22494, 22505, 22509, 22577, 22608, 22655,
22666, 22672, 22703, 22715, 22787, 22802, 22836, 22858, 22860, 22894, 22897, 22930, 23015,
23178, 23208, 23262, 23262, 23281, 23290, 23366, 23373, 23390, 23392, 23411, 23417, 23422,
23503, 23543, 23561, 23569, 23569, 23586, 23607, 23609, 23726, 23731, 23792, 23800, 23868,
23890, 23904, 23929, 23953, 23974, 24028, 24032, 24033, 24109, 24111, 24120, 24180, 24257,
24276, 24344, 24347, 24362, 24401, 24438, 24492, 24493, 24525, 24539, 24586, 24610, 24618,
24683, 24697, 24699, 24702, 24702, 24709, 24732, 24737, 24746, 24783, 24795, 24820, 24829,
24858, 24875, 24886, 24922, 24949, 24953, 24956, 24980, 25010, 25088, 25090, 25104, 25250,
25304, 25306, 25324, 25324, 25360, 25448, 25489, 25560, 25568, 25603, 25606, 25613, 25634,
25652, 25705, 25725, 25726, 25754, 25795, 25853, 25906, 25954, 25961, 26036, 26045, 26096,
26098, 26100, 26146, 26164, 26191, 26207, 26257, 26263, 26292, 26317, 26349, 26352, 26381,
26443, 26445, 26459, 26511, 26515, 26544, 26774, 26809, 26815, 26819, 26820, 26829, 26843,
26941, 26962, 26972, 26978, 26994, 26994, 27110, 27155, 27211, 27229, 27291, 27311, 27323,
27392, 27434, 27473, 27475, 27514, 27518, 27518, 27528, 27541, 27585, 27617, 27636, 27645,
27660, 27675, 27698, 27701, 27713, 27719, 27756, 27761, 27797, 27827, 27887, 27963, 27973,
28025, 28026, 28033, 28051, 28054, 28063, 28122, 28124, 28228, 28255, 28280, 28283, 28328,
28355, 28386, 28406, 28432, 28470, 28472, 28487, 28515, 28536, 28611, 28626, 28645, 28671,
28705, 28715, 28749, 28751, 28763, 28772, 28795, 28803, 28836, 28872, 28973, 28986, 29007,
29007, 29035, 29081, 29102, 29128, 29147, 29167, 29188, 29218, 29223, 29241, 29381, 29416,
29453, 29465, 29479, 29499, 29506, 29518, 29546, 29546, 29620, 29629, 29654, 29684, 29737,
29784, 29805, 29814, 29819, 29851, 29857, 29866, 29877, 29907, 29939, 29993, 29994, 30036,
30066, 30067, 30088, 30094, 30149, 30150, 30202, 30227, 30249, 30266, 30311, 30326, 30375,
30466, 30471, 30474, 30509, 30528, 30614, 30614, 30630, 30647, 30694, 30700, 30748, 30802,
30805, 30812, 30826, 30845, 30873, 30879, 30930, 30982, 31057, 31069, 31073, 31093, 31105,
31122, 31193, 31231, 31236, 31278, 31283, 31290, 31292, 31331, 31336, 31463, 31480, 31490,
31498, 31499, 31578, 31603, 31603, 31611, 31627, 31693, 31719, 31758, 31792, 31815, 31819,
31828, 31890, 31924, 31950, 32014, 32081, 32103, 32107, 32112, 32114, 32116, 32131, 32164,
32195, 32207, 32247, 32254, 32284, 32289, 32322, 32402, 32417, 32457, 32474, 32482, 32525,
32583, 32616, 32709, 32761, 32762, 32774, 32799, 32857, 32882, 32892, 32915, 32974, 33049,
33118, 33419, 33437, 33456, 33458, 33466, 33479, 33480, 33493, 33521, 33521, 33529, 33533,
33541, 33549, 33555, 33582, 33602, 33622, 33638, 33642, 33642, 33683, 33726, 33735, 33749,
33755, 33790, 33798, 33820, 33825, 33892, 33893, 33920, 33938, 33966, 33975, 33993, 34003,
34009, 34035, 34098, 34105, 34156, 34171, 34232, 34236, 34237, 34259, 34261, 34269, 34299,
34328, 34330, 34359, 34367, 34396, 34403, 34414, 34441, 34459, 34547, 34570, 34643, 34683,
34753, 34801, 34834, 34846, 34867, 34885, 34931, 34941, 34949, 34965, 34980, 35003, 35023,
35043, 35101, 35132, 35132, 35163, 35209, 35227, 35314, 35324, 35372, 35381, 35397, 35561,
35614, 35636, 35654, 35657, 35691, 35692, 35692, 35710, 35745, 35781, 35836, 35868, 35934,
35939, 36005, 36018, 36065, 36068, 36075, 36076, 36085, 36144, 36153, 36153, 36168, 36169.

TABLE 3
particularly preferred SEQ ID NOs in the sequence listing

| SEQ | |
|---|---|
| ID NO: | Further details |
| 2542 | This MafB-related protein has a 13mer leader peptide (with |
| | a GTG start codon), which can be removed post-translationally |
| | to provide a N-terminal cysteine. |
| 11994 | This thiosulphate sulphur transferase has a 19mer leader |
| | peptide, which can be removed post-translationally to provide |
| | a N-terminal cysteine. |
| 13348 | This haemoglobin-haptoglobin-utilization protein A has a 17mer |
| | leader peptide, which can be removed post-translationally to |
| | provide a N-terminal cysteine. |
| 13958 | This protein has a non-ATG start codon. |
| 14015 | — |
| 14530 | This protein has a 18mer leader peptide which can be removed. |
| 14709 | This protein has a 19mer leader peptide, which can be removed |
| | post-translationally to provide a N-terminal cysteine. |
| 14917 | This hemoglobin-haptoglobin utilization protein B has a 22mer |
| | leader peptide. |
| 14994 | — |
| 15162 | — |
| 15624 | This protein has a 19mer leader peptide which can be removed. |
| 15660 | This haemoglobin-haptoglobin-utilization protein has a 17mer |
| | leader peptide, which can be removed post-translationally to |
| | provide a N-terminal cysteine. |
| 15731 | — |
| 15955 | An outer membrane autotransporter barrel domain protein |
| 18039 | This protein has a TTG start codon. |
| 18256 | This protein is a hemagglutinin. |
| 18327 | This membrane protein is pilus-related. |
| 18491 | This protein has a 15mer leader peptide which can be removed. |
| 18563 | This protein has a TTG start codon. |
| 18722 | This peptidase has a 19mer leader peptide, which can be |
| | removed post-translationally to provide a N-terminal cysteine. |
| 18794 | — |
| 18976 | This protein is a hemolysin. |
| 19438 | — |
| 19570 | — |
| 19658 | — |
| 20742 | This is an outer membrane protein. |
| 20850 | This protein raises good anti-bactericidal antibodies. |
| 21500 | This protein has a TTG start codon. |
| 21873 | — |
| 24153 | This protein is a HAD-superfamily hydrolase. |
| 24810 | This protein is periplasmic |
| 25201 | — |
| 25368 | — |
| 25422 | This protein, with a GTG start codon, is hemolysin-like |
| 25433 | — |
| 25495 | — |
| 25596 | This protein has a TTG start codon and a 92% identical |
| | gonococcal homolog |
| 25615 | — |
| 28470 | This lipoprotein has a 18mer leader peptide, which can |
| | be removed post-translationally to provide a N-terminal |
| | cysteine. It is likely an adhesin. |
| 28653 | This protein has a GTG start codon. |
| 29557 | This is a membrane-bound protein |
| 29817 | This is a periplasmic protein |
| 32279 | — |
| 32363 | — |
| 32380 | — |
| 32436 | — |
| 32443 | This is a fimbrial protein with a 30mer leader peptide |
| | which can be removed. |
| 32562 | — |
| 32588 | — |
| 32616 | This lipoprotein has a 17mer leader peptide, which can be |
| | removed post-translationally to provide a N-terminal |
| | cysteine. |
| 32675 | — |
| 32708 | — |
| 32736 | This antioxidant protein has a 16mer leader peptide which can be |
| | removed. |
| 32815 | This protein binds extracellularly to lactoferrin. |
| 32856 | This MafB-related protein has a 15mer leader peptide. |
| 32895 | — |
| 32955 | — |
| 33068 | — |
| 33327 | — |
| 33368 | This protein has a 15mer leader peptide which can be removed. |
| 33638 | This is a lipoprotein. |
| 33646 | — |
| 33651 | — |
| 33652 | This protein has a GTG start codon. |
| 33676 | — |
| 33715 | — |
| 33857 | — |
| 33934 | This is a membrane protein, likely in the inner membrane. |
| 33989 | — |
| 34094 | This protein has a TTG start codon. |
| 36176 | — |
| 36177 | — |
| 36178 | — |

1. A polypeptide comprising an amino acid sequence that has at least 75% sequence identity to one or more of SEQ ID NOs: 1 to 36178 and 72357 to 72990.

2. The polypeptide of claim 1, comprising one or more of amino acid sequences SEQ ID NOs: 1 to 36178 and 72357 to 72990.

3. A polypeptide comprising a fragment of at least 7 consecutive amino acids from one or more of SEQ ID NOS: 1 to 36178 and 72357 to 72990.

4. The polypeptide of claim 3, wherein the fragment comprises a T-cell or a B-cell epitope from the SEQ ID NO: amino acid sequence.

5. An antibody that binds to the polypeptide of claim 1.

6. The antibody of claim 5, wherein the antibody is a monoclonal antibody.

7. A nucleic acid comprising a nucleotide sequence that has at least 75% sequence identity to one or more of SEQ ID NOS: 36179 to 72356.

8. The nucleic acid of claim 7, comprising an nucleotide sequence selected from SEQ ID NOS: 36179 to 72356.

9. A nucleic acid that can hybridize to the nucleic acid of claim 8 under high stringency conditions.

10. A nucleic acid comprising a fragment of 10 or more consecutive nucleotides from one or more of SEQ ID NOS: 36179 to 72356.

11. A nucleic acid encoding the antibody of claim 5.

12. A composition comprising: (a) the polypeptide of claim 1; and (b) a pharmaceutically acceptable carrier.

13. The composition of claim 12, further comprising a vaccine adjuvant.

14. A method of treating a patient, comprising administering to the patient a therapeutically effective amount of the composition of claim 12.

15. The method of claim 14 for preventing bacterial meningitis (in particular, meningococcal meningitis).
